(19) United States
(12) Patent Application Publication
(10) Pub. No.: US 2007/0213640 A1
(43) Pub. Date: Sep. 13, 2007

(54) Measurement aid for digital cervix examination

(76) Inventors: Hebah Noshy Mansour, La Mirada, CA (US); Ramez Emile Necola Shehada, La Mirada, CA (US)

Correspondence Address: HEBAH NOSHY MANSOUR, 14759 HARDAWAY DR., LA MIRADA, CA, 90638, US

(21) Appl. No.: 11/712,086

(22) Filed: Feb. 26, 2007

Related U.S. Application Data
(60) Provisional application No. 60/777,248, filed on Feb. 26, 2006.

(51) Int. Cl. A61B 5/103; A61B 5/117

(52) U.S. Cl. 600/591; 600/588

(57) ABSTRACT

The present invention is directed to a system and method for monitoring the dilatation of the uterine cervix during labor and deliver. The system has a gauge and a monitoring unit. In one embodiment the gauge includes features for convenient attachment to the gloved fingertips of the clinician performing a routine digital probing of the cervix. The monitoring unit includes a sensor to automatically read the measurement of the gauge and a processor to process, record and display the sensor data. This system may be valuable for the assessment of the progress of labor and the early detection of delivery complications.

### CROSS-REFERENCE TO RELATED APPLICATION(S)

This Application claims the benefit of U.S. Provisional Application No. 60/777,248, filed Feb. 26, 2006, entitled Measurement Aid for Digital Cervix Examination, the entire contents of which are incorporated herein by reference.

### FIELD OF THE INVENTION

The present invention relates to a system and method for monitoring the dilatation of the uterine cervix during labor and delivery, in particular, a system and method utilizing a gauge that adheres to the gloved fingertips of a clinician for measuring the diameter of the cervical opening.

### BACKGROUND OF THE INVENTION

The diameter of the opening of the uterine cervix (or cervical diameter, used interchangeably herein) increases during the final stages of pregnancy to facilitate the birth process and enable the delivery of the fetus. The cervical diameter is considered an important obstetrical indicator and therefore, its accurate measurement may be valuable for the assessment of the progress of labor and the early detection of delivery complications. The measurement of the cervical diameter may affect the decision of whether to wait for a normal delivery, to administer a labor inducing drugs, or to perform a C-section delivery.

Current clinical practice for measuring the cervical diameter is performed manually by inserting a gloved hand into the vagina and then using the middle and index fingers to probe the diameter of the cervix. The fingertips are used to palpate and locate the cervical Os and the fingers are then spread apart such that their tips are in contact with the opposite edges of the cervical opening. The degree of fingers spread may be used to assess the distance between the fingertips, which corresponds to the diameter of the cervix opening.

This digital probing method is approximate and its accuracy is primarily dependent on the experience of the examiner in estimating the cervical diameter using fingers. This type of measurement is not repeatable even by the same examiner. The accuracy may be further degraded by interobserver variability if different clinicians examine the patient during the course of labor. One examiner cannot reliably communicate that degree of dilation to another examiner without some objective scale. Such inconsistent measurements of the cervix diameter may hinder the early detection of dysfunctional labor and delivery complications.

There have been many attempts to develop devices for the accurate and user-independent measurement of the cervical diameter. However, these devices have failed to gain wide clinical acceptance due to several limitations, including the complexity of use, inaccuracy of measurements, tissue trauma caused by the devices or their components, costly sterilization between uses, and/or patient discomfort.

Consequently, the digital probing method continues to be a favored method of measuring the cervical diameter. Therefore, there is a need to improve the accuracy of the digital probing method, reduce its interobserver variability, and enable a semi-automated recording of its measurements for the calculation of the cervical dilatation rate and the generation of a partogram.

### SUMMARY OF THE INVENTION

The present invention discloses a system and method for measuring the dilatation of the uterine cervix (or cervix, used interchangeably herein) during labor and delivery utilizing the routine digital probing method.

The system is comprised of an attachable gauge and a monitoring unit. The gauge may include an indicator to display its extension and a means to adhere to the glove worn by the clinician during digital probing of the cervix. The indicator of the gauge may be visually readable to the clinician and/or machine readable by the monitoring unit.

The application and utilization of the disclosed gauge may not require any additional effort, training or skills beyond that required for routine digital probing of the cervix. The gauge may be attached directly to the glove covering the two probing fingers of the clinician performing the cervix examination.

In one embodiment, the indicator of the gauge may be a string that is configured in a zigzag or sawtooth pattern which changes appearance depending on the extension of the gauge. In another embodiment, the string may be arranged in a star-shaped pattern which also changes appearance depending on the extension of the gauge.

The monitoring unit may include a sensor (e.g. a CCD or a barcode reader) to read the gauge indicator and acquire its measurement. The monitoring unit may also include a processor to calculate the dilatation rate and construct partogram from a series of consecutively measured cervical diameters. The monitoring unit may also include a display screen to display the cervical diameter, the dilatation rate, and the partogram. Furthermore, the monitoring unit may be used to setoff an alert to remind to the clinician to digitally probing of the cervix.

In one method, the gauge may be attached to gloved probing fingers of the clinician, the probing fingers inserted into the vagina and spread apart to touch the rim of the cervical opening, the probing fingers withdrawn from the vagina, and the clinician reading the extension indicator of the gauge to determine the cervical diameter. In another method, a monitoring unit may be used to automatically read the indicator of the gauge and record its measurement to determine the cervical diameter and estimate the dilatation rate of the cervix from sequential measurements of the cervical diameter.

### BRIEF DESCRIPTION OF THE DRAWINGS

FIG. 1 shows an embodiment of a gauge attached to gloved fingertips that are positioned to measure cervical diameter in accordance with the present invention.

FIG. 2A shows a top view of the gauge of FIG. 1.

FIG. 2B shows a side view of the gauge of FIG. 1.

FIG. 3A shows a gauge indicator reading a 3-cm cervical diameter.

FIG. 3B shows a gauge indicator reading a 5-cm cervical diameter.

FIG. 3C shows a gauge indicator reading a 8-cm cervical diameter.

FIG. 4 shows another embodiment of the gauge.

FIG. 5A shows another embodiment of the gauge indicator with a star pattern.

FIG. 5B shows another embodiment of the gauge indicator with a fan pattern.

FIG. 5C shows another embodiment of the gauge indicator with a sinusoidal pattern.

FIG. 5D shows another embodiment of the gauge indicator with a coil pattern.

FIG. 5E shows another embodiment of the gauge indicator with an uneven sinusoidal pattern.

FIG. 5F shows a reflectance signal generated by optically scanning the uneven sinusoidal pattern of FIG. 5E.

FIG. 6 shows an embodiment of a monitoring unit in accordance with the present invention.

FIG. 7 shows an embodiment of a display screen of a monitoring unit in accordance with the present invention.

### DETAILED DESCRIPTION OF THE INVENTION

The cervix monitoring system is comprised of a gauge and a monitoring unit. The gauge may be manually used to measure the cervical diameter using the routine digital probing method. The monitoring unit may be used to automatically read the measurement of the gauge, record consecutive cervical diameter measurements, calculate the cervical dilatation rate, and display a partogram.

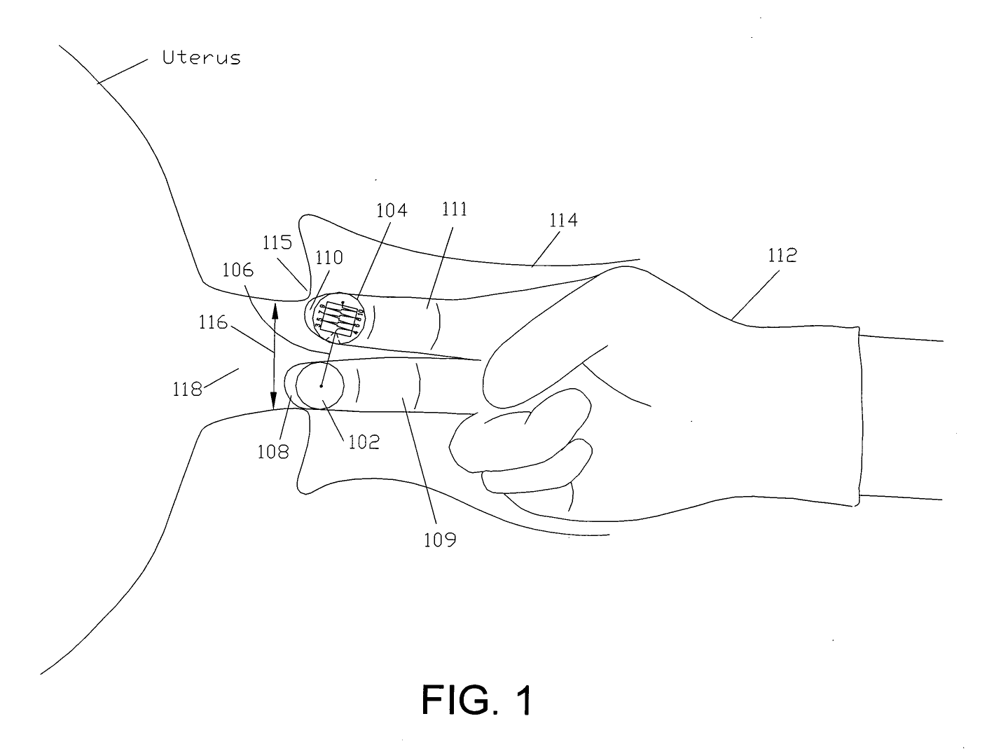

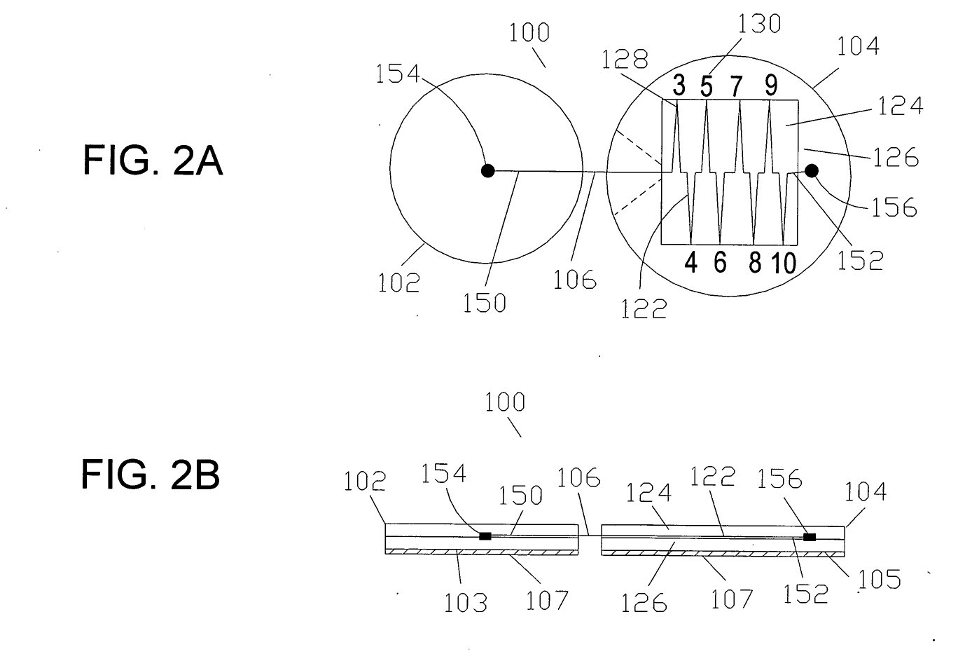

A preferred embodiment of the disposable gauge 100 is shown in FIG. 1 and FIG. 2. The gauge 100 may be composed of an anchor patch 102 and an indicator patch 104 that are interconnected with a measurement string 106.

The patches 102 and 104 are preferably thin and flexible and may be made of a soft material such as, for example, polyethylene, nylon, or silicone. The patches 102 and 104 may include an embedded fiber mesh (not shown) for structural reinforcement and to provide strong attachment points to the endings 150 and 152 of the string 106. The patches 102 and 104 may have a typical diameter of about 1.5-cm while the string 106 may have a typical length of about 10-cm. The patches 102 and 104 may have a back surface 103 and 105 covered with an adhesive material 107 to enable their attachment to the material of the examination glove as described below.

The adhesive material 107 may be of any kind that would allow a fast and a firm attachment of the patches 102 and 104 to the material of the examination glove. The adhesive 107 may be a pressure sensitive adhesive (PSA).

The patch 102 may provide an anchor point 154 for the ending 150 of the string 106. The indicator patch 104 may provide an anchor point 156 for the ending 152 of the string 106 but it may also include an additional portion of the string 106 packed in a zigzag or sawtooth pattern 122 between an optically transparent window 124 and a reflective background sheet 126.

The string may be arranged in the zigzag pattern 122 and stabilized in that form using a thin layer of a transparent pressure sensitive adhesive (not shown) placed between the transparent window 124 and a reflective background sheet 126. The pressure sensitive adhesive (not shown) stabilizes the string 106 to maintain its zigzag pattern 122 without preventing the string 106 from being pulled out of the zigzag pattern 122 when the anchor patch 102 is moved away from the indicator patch 104.

The string 106 may be selected of a color that is highly contrasting with the color of the reflective background sheet 126. For example, the color of the string 106 may be matte black, while the color of the reflective background sheet 126 may be bright white.

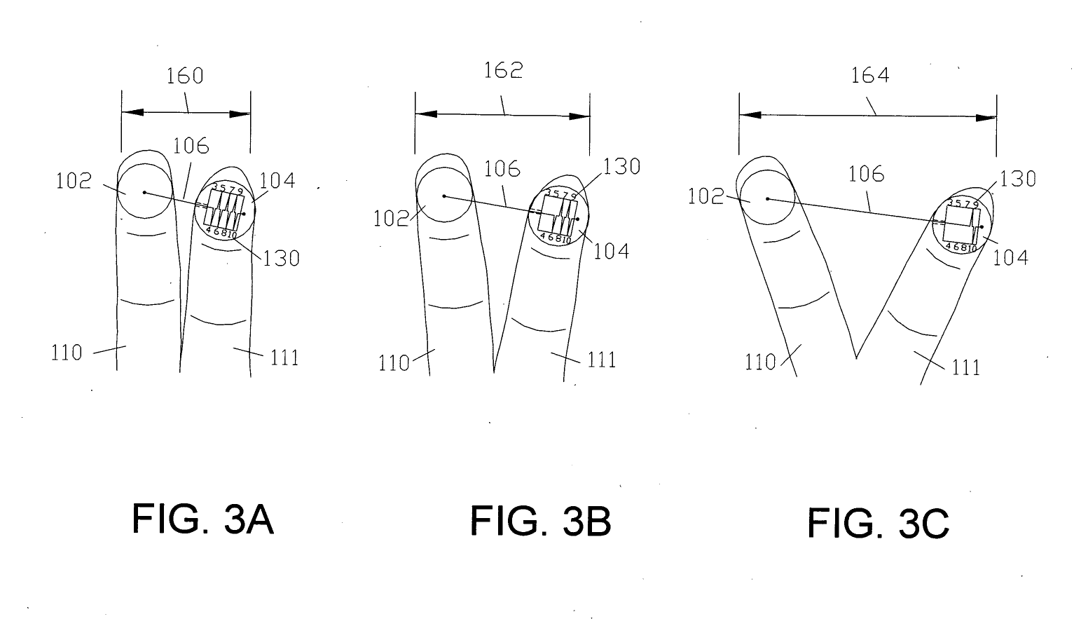

The design and dimensions of the zigzag pattern 122 may be configured such that pulling out the string 106 from the indicator patch 104 may cause the sequential disappearance of the corners 128 of the zigzag pattern 122. Each zigzag corner 128 may have an adjacent number 130 imprinted on the transparent window 124 or the reflective background sheet 126. The largest numeral from the set 130 which does not have a zigzag corner 128 pointing to it represents the distance between the fingertips or the diameter 116 as illustrated by the example shown in FIG. 3.

The gage 100 in FIG. 3A shows that the zigzag corner pointing to number 3 is missing which indicates that the distance 160 between the fingertips is about 3-cm. The gage 100 in FIG. 3B shows that all the zigzag corners pointing to all numbers up to 5 are missing which indicates that the distance 160 between the fingertips is about 5-cm. The gage 100 in FIG. 3C shows that all the zigzag corners pointing to all numbers up to 8 is missing which indicates that the distance 160 between the fingertips is about 8-cm.

In a typical application, the user may wear an examination glove and press the anterior fingertip 108 of the middle finger 109 and the anterior fingertip 110 of the index finger 111 against the adhesive-covered back surface 103 of the patches 102 and the back surface 105 of the patch 104, respectively. The patches 102 and 104 become attached to the anterior (or palmer) fingertips 108 and 110 of the fingers 109 and 111 of the gloved examiner hand 112, respectively as shown in FIG. 2.

The gloved fingers 109 and 111 may be inserted into the vagina 114 and spread opened until their tips 108 and 110 contact (or palpate) the lips (or rim) of the cervix 115 to measure the diameter 116 of the cervix opening 118. The spreading action of the fingers 109 and 111 moves the patches 102 and 104 away from each other thereby pulling the string 106 out of the indicator patch 104, which indicates the length of the pulled-out string as described below. The length of the pulled-out string corresponds to the distance between the fingertips 108 and 112 or equivalently the diameter 116 of the cervical opening 118.

Alternative to the numbers 130, other representing alphanumeric symbols, color-coded spots, drawings, or measurement tick marks may be used.

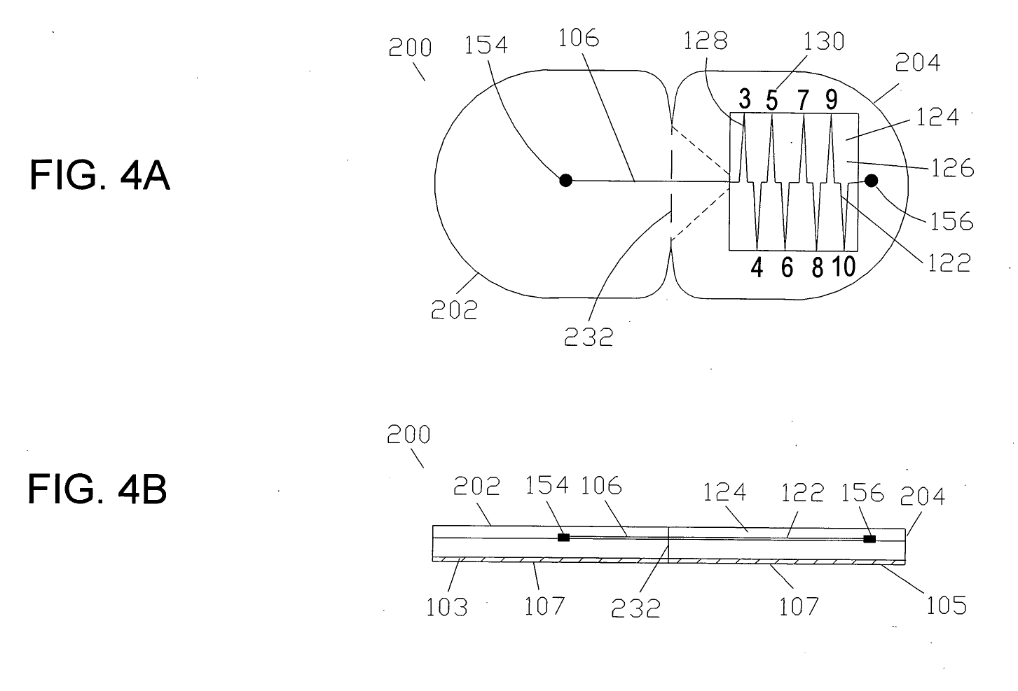

An alternative embodiment of the disposable gauge is shown in FIG. 4. The gauge 200 may have an anchor patch 202 and an indicator patch 204 that are initially connected together by a perforated line of separation 232. The patches 202 and 204 may separate along the line of separation 232 only upon an intentional forcible opening of the fingers 109 and 111. The initial force needed to separate anchor patch 202 from the indicator patch 204 may be adjusted by the shape and number of the perforations along the separation line 232. This configuration may safeguard against unintentional opening of the fingers 109 and 111 prior to their proper positioning on the lips (or rim) of the cervix 115.

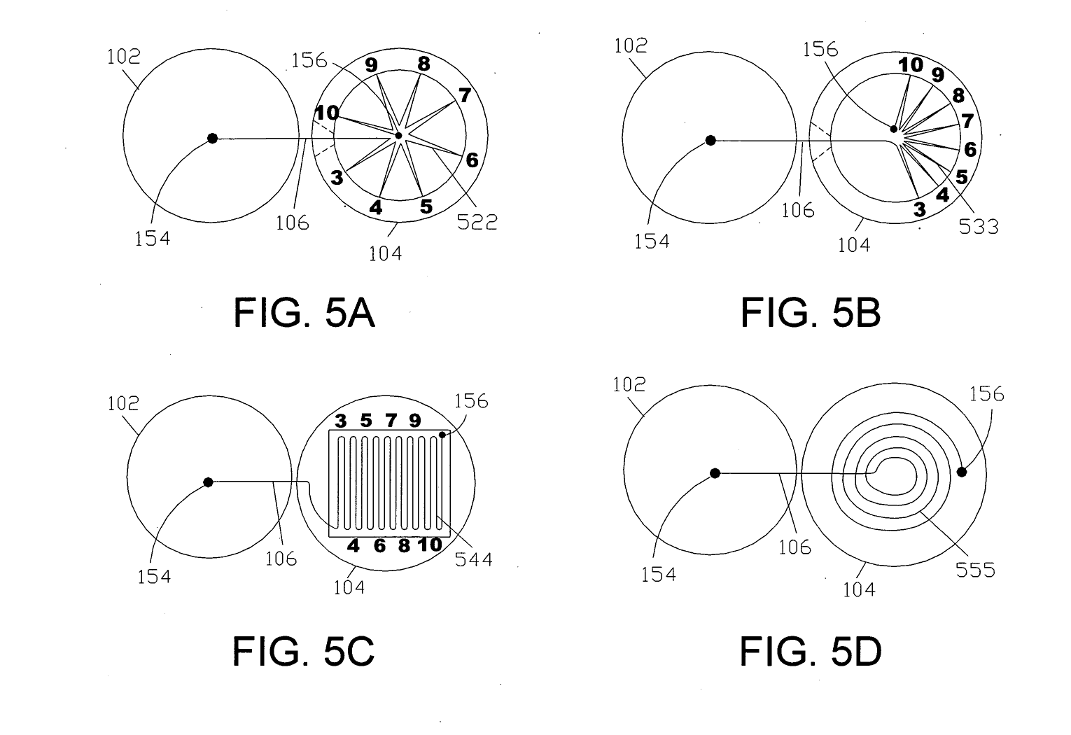

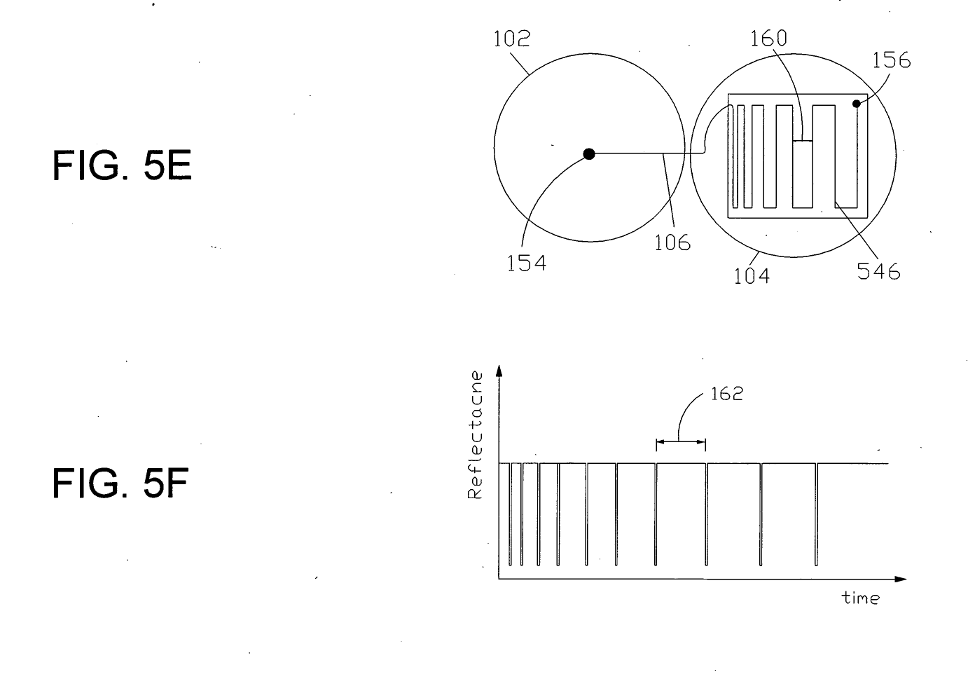

Alternative gauge embodiments may utilize geometrical patterns other than the zigzag pattern 122 such as, for example, the star pattern 522 shown in FIG. 5A, the fan pattern 533 shown in FIG. 5B, the sinusoidal pattern 544 shown in FIG. 5C, and the coil pattern 555 shown in FIG. 5D. The string 106 arranged in the uneven sinusoidal pattern 546 shown in FIG. 5E may have unequal separation distances 160 between its turns. These unequal distances between the turns of the sting 106 may produce electrical pulses of unequal timing 162 as shown in FIG. 5F when scanned by an optical reflectance sensor such as a barcode reader. The distances 160 may be configured to generate a timing 162 that is representative of the distance between the fingertips or the diameter 116.

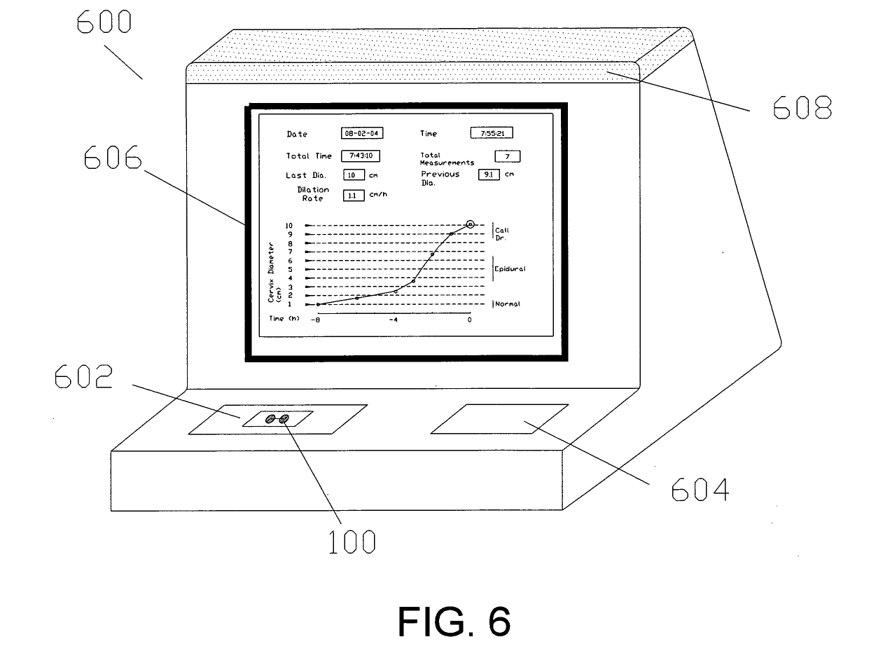

The monitor 600 shown in FIG. 6 may include an automatic gauge dispenser 602, a gauge reader 604, a display screen 606, a measurement reminder light 608, and a processor (not shown).

The automatic gauge dispenser 602 may utilize a bilayer roll of non-stick tape (not shown) with the gauges 100 sandwiched in-between the two layers of the tape. The tape may be automatically advanced and its layers separated to offer the user one gauge at a time. The gauge 100 may be offered with its adhesive covered surfaces 103 and 105 facing upwards.

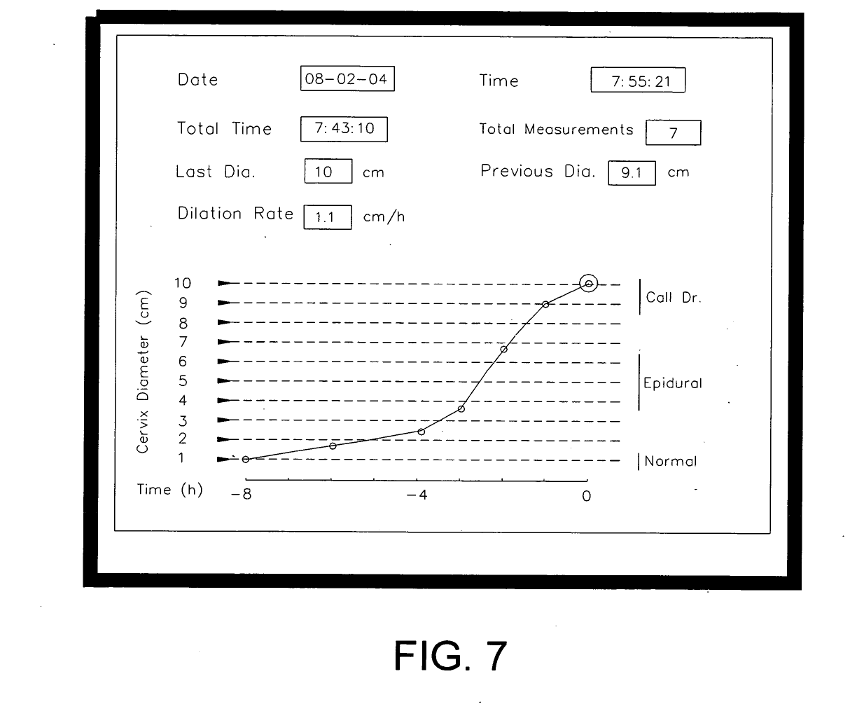

The gauge reader 604 may be used to automatically read the measurement of the gauge 100. The machine reader may utilize barcode reading technology; fingerprint reading technology, or optical imaging with image recognition techniques. The reading technology or the image analysis method will depend on the type of the geometrical pattern used in the indicator patch 104 of the gauge 100. The gauge reader 604 transmits its reading to the processor which processes the diameter measurement with the time of the reading and compares it to previous diameter-time measurements to calculate the current dilatation rate and generate a partogram. The partogram is a graphical display of the cervical dilation versus time and may be used for the assessment of labor. The processor may display the information on the display screen 606. A detailed view of the display screen is shown in FIG. 7.

The processor may also trigger a reminder light 608 to alert the attending clinician to take a measurement of the cervical diameter. The light 608 may be turned on at a preselected time interval following the last reading to attract attention of the attending clinician that a new measurement is due to be taken. The reminder alert may be preferably of the silent visual type such as the reminder light 608. However, reminder alert may be also of the audio type. The alert may be also an electronic signal transmitted to the local nursing station of the Labor and delivery ward.

What is claimed is:

1. A gauge for measuring the diameter of the uterine cervix comprising:
a first patch attachable to a first gloved fingertip;
a second patch attachable to a second gloved fingertip;
a string between the first patch and the second patch;
an indicator in the first patch indicating the distance between the first patch and the second patch.

2. The gauge of claim 1, wherein the indicator is configured to display a segment of the string arranged in a geometrical pattern.

3. The gauge of claim 1, wherein the indicator is configured to display a segment of the string arranged in a geometrical pattern superimposed with alphanumerical characters.

4. The gauge of claim 1, wherein the indicator is configured to display a segment of the string arranged in a geometrical pattern selected from the group comprising: zigzag, sawtooth, sinusoidal, fan-shaped, coil-shaped, and star-shaped.

5. The gauge of claim 1, wherein the means for attaching is an adhesive.

6. The gauge of claim 1, wherein the means for attaching is a pressure sensitive adhesive.

7. The gauge of claim 1, wherein the gauge is made of a flexible material.
